(19) United States
(12) Patent Application Publication
(10) Pub. No.: US 2009/0093844 A1
(43) Pub. Date: Apr. 9, 2009

(54) Elastic covered dynamic stabilization connector and assembly

(76) Inventor: Roger P. Jackson, Prairie Village, KS (US)

Correspondence Address: LAW OFFICE OF JOHN C. MCMAHON, P.O. BOX 30069, KANSAS CITY, MO, 64112, US

(21) Appl. No.: 12/316,131

(22) Filed: Dec. 9, 2008

Related U.S. Application Data
(63) Continuation-in-part of application No. 11/522,503, filed on Sep. 14, 2006.
(60) Provisional application No. 61/009,228, filed on Dec. 27, 2007.
(60) Provisional application No. 60/722,300, filed on Sep. 30, 2005.
(60) Provisional application No. 60/725,445, filed on Oct. 11, 2005.
(60) Provisional application No. 60/728,912, filed on Oct. 21, 2005.
(60) Provisional application No. 60/736,112, filed on Nov. 10, 2005.
(60) Provisional application No. 60/832,644, filed on Jul. 21, 2006.

(51) Int. Cl. A61B 17/70; A61B 17/04

(52) U.S. Cl. 606/254; 606/246; 606/264; 606/301; 606/308

(57) ABSTRACT

A dynamic fixation medical implant having at least two bone anchors includes a dynamic longitudinal connector having a solid core with an elastic over-molded portion. The over-molded portion may be firmly adhered or clamped to at least a portion of the solid core. Alternatively, the over-molded portion may completely envelope the solid core and in some embodiments slide on the core. At least one bone anchor includes at least one insert for gripping the over-molded portion without crushing such portion into fixed relation with the core.

### CROSS-REFERENCE TO RELATED APPLICATIONS

This application claims the benefit of U.S. Provisional Patent Application Ser. No. 61/009,228, filed Dec. 27, 2007, which is incorporated by reference herein. This application is also a continuation-in-part of U.S. patent application Ser. No. 11/522,503 filed Sep. 14, 2006 that claims the benefit of the following U.S. Provisional Applications: No. 60/722,300 filed Sep. 30, 2005; No. 60/725,445 filed Oct. 11, 2005; No. 60/728,912 filed Oct. 21, 2005; No. 60/736,112 filed Nov. 10, 2005; and No. 60/832,644 filed Jul. 21, 2006; all of which are incorporated by reference herein.

### BACKGROUND OF THE INVENTION

The present invention is directed to dynamic fixation assemblies for use in bone surgery, particularly spinal surgery, and in particular to longitudinal connectors and cooperating bone anchors or fasteners for such assemblies, the connectors being attached to at least two bone anchors.

Historically, it has been common to fuse adjacent vertebrae that are placed in fixed relation by the installation therealong of bone screws or other bone anchors and cooperating longitudinal connecting members or other elongate members. Fusion results in the permanent immobilization of one or more of the intervertebral joints. Because the anchoring of bone screws, hooks and other types of anchors directly to a vertebra can result in significant forces being placed on the vertebra, and such forces may ultimately result in the loosening of the bone screw or other anchor from the vertebra, fusion allows for the growth and development of a bone counterpart to the longitudinal connecting member that can maintain the spine in the desired position even if the implants ultimately fail or are removed. Because fusion has been a desired component of spinal stabilization procedures, longitudinal connecting members have been designed that are of a material, size and shape to largely resist bending (including flexion and extension), torsion, shear, distraction and compression, and thus substantially immobilize the portion of the spine that is to be fused. Thus, longitudinal connecting members are typically uniform along an entire length thereof, and usually made from a single or integral piece of material having a uniform diameter or width of a size to provide substantially rigid support in all planes.

Fusion, however, has some undesirable side effects. One apparent side effect is the immobilization of a portion of the spine. Furthermore, although fusion may result in a strengthened portion of the spine, it also has been linked to more rapid degeneration and even hyper-mobility and collapse of spinal motion segments that are adjacent to the portion of the spine being fused, reducing or eliminating the ability of such spinal joints to move in a more normal relation to one another. In certain instances, fusion has also failed to provide pain relief.

An alternative to fusion and the use of more rigid longitudinal connecting members or other rigid structure has been a “soft” or “dynamic” stabilization approach in which a flexible loop-, S-, C- or U-shaped member or a coil-like and/or a spring-like member is utilized as an elastic longitudinal connecting member fixed between a pair of pedicle screws in an attempt to create, as much as possible, a normal loading pattern between the vertebrae in flexion, extension, distraction, compression, side bending and torsion. Problems may arise with such devices, however, including tissue scarring, lack of adequate spinal support or being undesirably large or bulky when sized to provide adequate support, and lack of fatigue strength or endurance limit. Fatigue strength has been defined as the repeated loading and unloading of a specific stress on a material structure until it fails. Fatigue strength can be tensile or distraction, compression, shear, torsion, bending, or a combination of these.

Another type of soft or dynamic system known in the art includes bone anchors connected by flexible cords or strands, typically made from a plastic material. Such a cord or strand may be threaded through cannulated spacers that are disposed between adjacent bone anchors when such a cord or strand is implanted, tensioned and attached to the bone anchors. The spacers typically span the distance between bone anchors, providing limits on the bending movement of the cord or strand and thus strengthening and supporting the overall system. Such cord or strand-type systems require specialized bone anchors and tooling for tensioning and holding the cord or strand in the bone anchors. Although flexible, the cords or strands utilized in such systems do not allow for any significant elastic distraction of the system once implanted because the cord or strand must be stretched or pulled to maximum or near maximum tension in order to provide a stable, supportive system. Also, as currently designed, these systems do not provide any significant torsional resistance. Furthermore, such systems in certain embodiments can allow for a pulling away of the adjacent bone screws from the cannulated spacers providing space for the growth of soft tissue that may result in pinching, scarring and resultant pain to the patient and may affect the performance of the system.

The complex dynamic conditions associated with spinal movement therefore provide quite a challenge for the design of elongate elastic longitudinal connecting members that exhibit an adequate fatigue strength to provide stabilization and protected motion of the spine, without fusion, and allow for some natural movement of the portion of the spine being reinforced and supported by the elongate elastic connecting member. A further challenge are situations in which a portion or length of the spine requires a more rigid stabilization, possibly including fusion, while another portion or length may be better supported by a more dynamic system that allows for protected movement.

### SUMMARY OF THE INVENTION

A dynamic fixation medical implant with at least two bone anchors includes a dynamic longitudinal connecting member assembly having an inelastic core with an elastic outer portion. The outer portion may be over-molded or slid onto the core and then clamped and/or firmly adhered to at least a portion of the core. Alternatively, an over-molded portion may completely envelope the solid core and thus not be otherwise adhered or clamped to the core. At least one bone anchor grips the outer elastic portion without placing the elastic portion in fixed relation to the core at the bone anchor. Thus the bone anchors are movable toward and away from one another due to the elasticity of the outer elastic portion. Bone anchors for attaching to the outer elastic portion may include upper and lower inserts for even gripping about the elastic outer portion, thereby providing controlled and limited deformation of the polymer without crushing or overly compressing the polymer against the core, although other gripping options are possible.

### OBJECTS AND ADVANTAGES OF THE INVENTION

An object of the invention is to provide dynamic medical implant stabilization assemblies having longitudinal connecting members that include an elastic portion that allows for movement between connected bone anchors toward and away from one another. Another object of the invention is to provide such an elastic portion that is molded about and around a cooperating more inelastic and harder core portion such that the core portion provides support, including bending and shear resistance, while the elastic portion provides stress relief in the form of compression, distraction and torsional elasticity and resiliency, and wherein the bone anchor is directly attached to the elastic portion, the elastic portion being clamped and/or adhered to the core in some embodiments and being movable on the core surface in other embodiments. A further object of the invention is to provide dynamic medical implant longitudinal connecting members that may be utilized with a variety of bone screws (both fixed and polyaxial), hooks and other bone anchors. Another object of the invention is to provide a more rigid or solid connecting member portion or segment, if desired, such as a solid rod portion bonded or integral with the core portion that itself is surrounded by an elastomer. Additionally, it is an object of the invention to provide a lightweight, reduced volume, low profile assembly including at least two bone anchors and a longitudinal connecting member therebetween that in some cases can be cut to length. Furthermore, it is an object of the invention to provide apparatus and methods that are easy to use and especially adapted for the intended use thereof and wherein the apparatus are comparatively inexpensive to make and suitable for use.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is a side elevational view of a dynamic fixation connector according to the invention including a solid core having an inner extension shown in phantom and an over-molded polymer.

FIG. 2 is a reduced side elevational view of the connecting member of FIG. 1 shown with a pair of bone screws to form a dynamic connector assembly.

FIG. 3 is an enlarged front elevational view of the assembly of FIG. 2.

FIG. 4 is an enlarged and partial cross-sectional view taken along the line 4-4 of FIG. 3.

FIG. 5 is an enlarged and partial cross-sectional view, similar to FIG. 4 further showing details of one of the bone screws.

FIG. 6 is an enlarged and exploded perspective view of the connecting member of FIG. 1 and one of the bone screws of FIG. 2.

FIG. 7 is an enlarged and partial perspective view of the assembly of FIG. 2 with portions broken away to show the detail thereof.

FIG. 8 is a reduced and partial cross-sectional view taken along the line 8-8 of FIG. 2.

FIG. 9 is a reduced and partial cross-sectional view taken along the line 9-9 of FIG. 2.

FIG. 10 is a side elevational view of a second embodiment of a dynamic fixation connector according to the invention including a solid inner core shown in phantom and an over-molded, adhered polymer.

FIG. 11 is a side elevational view of a third embodiment of a dynamic fixation connector according to the invention including a solid inner core shown in phantom and a totally over-molded polymer.

FIG. 12 is a side elevational view of a fourth embodiment of a dynamic fixation connector according to the invention including a solid inner core with portions shown in phantom, an over-molded portion and a clamping sleeve.

FIG. 13 is an enlarged front elevational view of the clamping sleeve shown in FIG. 12.

FIG. 14 is a side elevational view of the dynamic fixation connector of FIGS. 1-9 shown with a pair of bone screws that have different closure tops and fewer inserts than the bone screws of FIGS. 2-9.

FIG. 15 is an enlarged and partial side elevational view of the connector and one of the bone screws of FIG. 14.

FIG. 16 is a side elevational view of the dynamic fixation connector of FIGS. 1-9 and 14-15 shown with a pair of bone screws that have different closure tops than the bone screws of FIGS. 2-9 and 14-15.

FIG. 17 is an enlarged and partial side elevational view of the connector and one of the bone screws shown in FIG. 16.

FIG. 18 is a side elevational view of a fifth embodiment of a dynamic fixation connector according to the invention including a solid inner core with portions shown in phantom, a slide-on elastic sleeve with portions shown in phantom and a clamping sleeve.

FIG. 19 is a cross-sectional view taken along the line 19-19 of FIG. 18.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of the longitudinal connectors of the application and cooperating bone anchors in actual use.

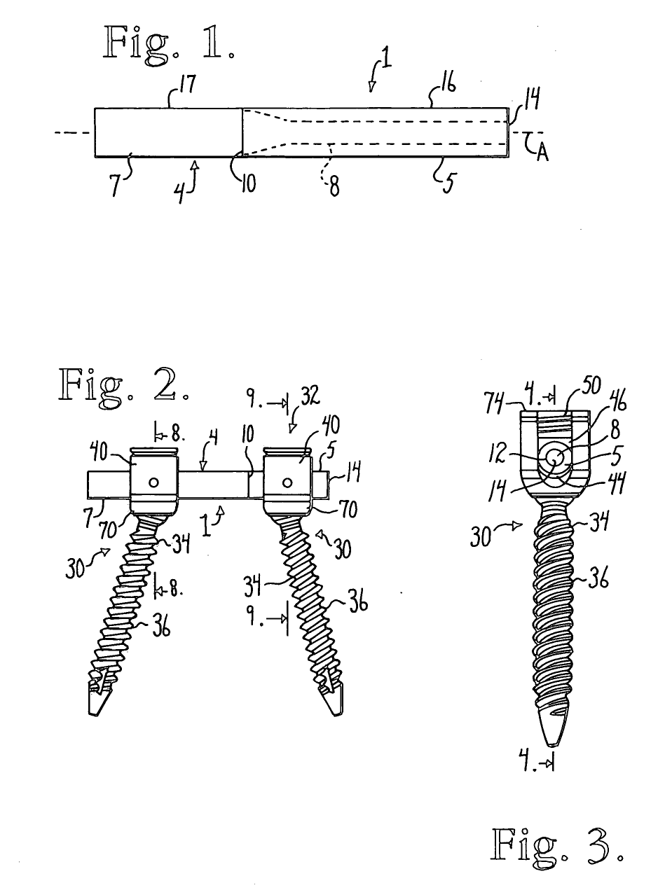

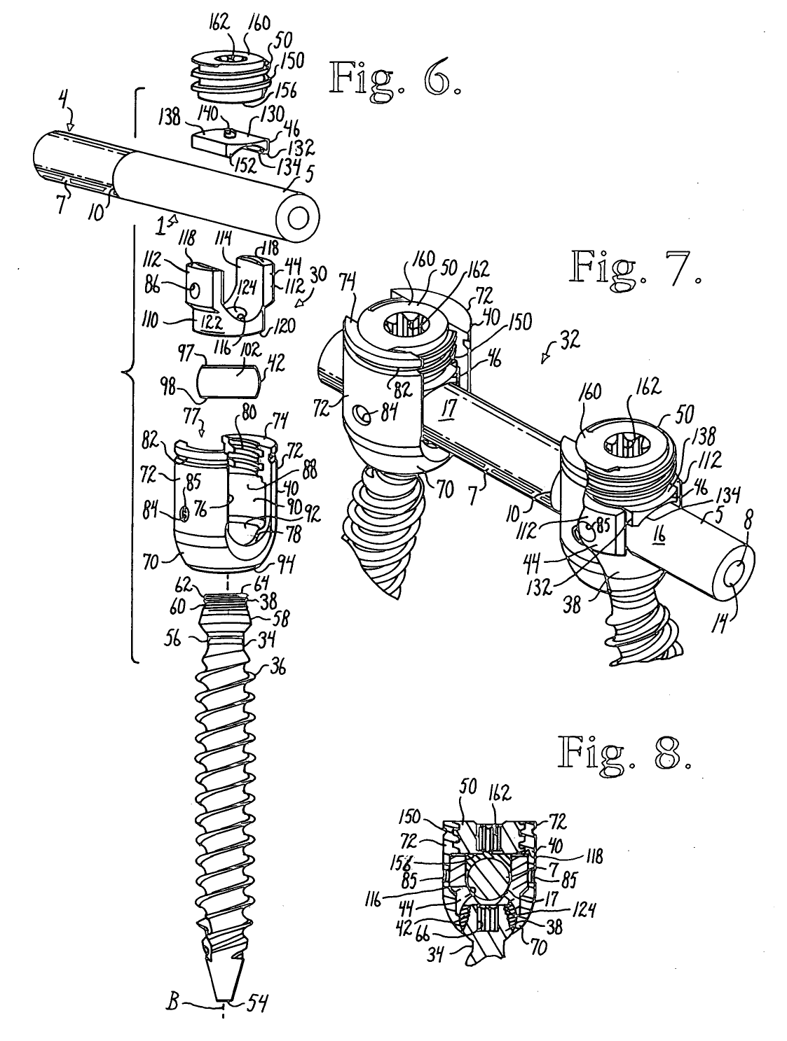

With reference to FIGS. 1-8, the reference numeral 1 generally designates a dynamic stabilization longitudinal connector according to the present invention. The connector 1 includes a substantially inelastic elongate segment, generally 4 and an elastic covering, illustrated by an over-mold 5 covering a portion of the segment 4. The illustrated elongate segment 4 further includes a first elongate portion 7 and an integral inner core extension 8 shown in phantom in FIG. 1. The extension 8 may also be a separate piece that is fixed to the portion 7. The elongate segment 4 extends along an entire central longitudinal axis A of the connector 1. In the illustrated embodiment, the connector 1 is cylindrical and of uniform circular cross-section along an entire length thereof. At a location 10, the cylindrical portion 7 tapers down to the core extension 8 of a smaller diameter than the portion 7. The over-molded portion 5 is molded and in some cases adhered onto the extension 8 starting at the location 10, extending along and about an outer surface 12 of the core extension 8 and in this embodiment ending flush to an end surface 14 of the extension 8. The portion 5 has an outer diameter defined at an outer surface 16 that is the same or substantially similar to the outer diameter of an outer surface 17 of the portion 7. It is foreseen that according to other embodiments of the invention, the portions 7 and 8 may be of constant cross-section and the over-molded polymer portion 5 may be of a greater outer diameter than the portions 7 and 8. The portions 5, 7 and 8 of connectors according to the invention may have other cross-sectional geometries, including but not limited to oval, square, rectangular and other polygonal shapes. Mixtures of cross-section may be utilized, for example, portions 5 and 7 may be of circular cross-section, having the same outer diameter, while the inner core extension 8 may be of square or rectangular cross-section.

The illustrated solid segment 4 of the longitudinal connector 1 illustrated in FIGS. 1-8 that includes the portion 7 with the tapered integral extension 8 is preferably made from metal, metal alloys or other suitable materials, including but not limited to stainless steel, titanium, titanium alloys and cobalt chrome. A preferred material for the segment 4 is a nickel titanium (NiTi, also commonly referred to by its trade name, Nitinol). The elongate segment 4 may also be made from plastic polymers such as pure and carbon reinforced polyetheretherketone (PEEK), ultra-high-molecular weight-polyethylene (UHMWP), polyurethanes and composites. The over-molded portion 5 is elastomeric and may be made of a variety of materials including plastics and composites. The illustrated portion 5 is a molded thermoplastic elastomer, made for example from polyurethanes, polyurethane blends or silicone-containing polymers. However, any suitable polymer material may be used for the over-molded portion 5. It is believed that certain polymers, such a silicone-containing polymers are advantageously chemically bonded to the surface of the segment 4 during molding thereon, particularly when the segment 4 is made from titanium with certain surface treatments and from polymers, such as PEEK. The over-molded polymer portion 5 may also be otherwise adhered to the segment 4, for example, with the aid of an adhesive when the segment 4 is made from a metal-containing substance such as Nitinol. As will be discussed in greater detail below, over-molded silicone-containing polymers may be relatively soft, elastic and compressible against the core 8, therefore bone anchor assemblies according to the invention advantageously include upper and lower pressure inserts, or closure top and insert combinations that press upon the portion 5 to a limited degree to produce a controlled amount of deformation in the polymer without locking the polymer against the core 8, allowing limited movement of bone anchor with respect to the core 8 when the portion 5 is being gripped by the bone anchor.

In the illustrated embodiment, the section 7 of the elongate segment 4 is substantially smooth and cylindrical with a uniform circular cross-section, sized and shaped to be received in a bone anchor. In some embodiments according to the invention, the section 7 may be longer than what is shown in the drawings so that the section 7 may be attached to two or more bone anchors. The over-molded portion 5 advantageously cooperates with the inner core extension 8 and portion 7 to provide a flexible or less stiff dynamic segment that allows for bending, torsion, compression and distraction of the connector 1. The over-molded portion 5 further provides a smooth substantially cylindrical surface that protects a patient's body tissue from damage that might otherwise occur with, for example, a spring-like dynamic member or a corded member wherein an outer spacer may be pulled away from a cooperating plate or bone screw surface. The molded portion 5 is fabricated about the inner core 8 as will be described more fully below, and in the presence of the segment 7, with molded plastic flowing about the core 8. It is noted that the portion 7 and the core 8 may be sized and made from such materials as to provide for a relatively more rigid or stiff connector 1 or a relatively more flexible connector 1 with respect to flex or bendability along the connector 1. Such flexibility therefore may be varied by changing the outer diameter of the core 8 and thus likewise changing the diametric size of the over-molded portion 5. Also, it is noted that longer connectors 1 may need to be stiffer and thus larger in diameter than shorter connectors 1 and/or made from different materials. In addition, since the distance between the bone screw assembly receivers or heads can vary, the connector may need to be more or less stiff.

The dynamic connector 1 cooperates with at least a pair of bone anchors, such as the polyaxial bone screws, generally 30 shown in FIGS. 2 and 6-8, providing an overall dynamic connection system, generally 32. The connector 1 is captured and fixed in place at the segment 7 as well as at the over-molded portion 5 by cooperation between the bone screws 30.

When the segment 7 is made from a rigid material, such as titanium, a variety of bone anchors may be utilized for receiving and fixing the segment 7 to bone, including, but not limited to fixed, monoaxial bone screws, hinged bone screws, polyaxial bone screws, and bone hooks and the like, with or without one or more compression inserts, that may in turn cooperate with a variety of closure structures having threads, flanges, or other structure for fixing the closure structure to the bone anchor, and may include other features, for example, break-off tops and inner set screws as well as associated pressure inserts. The illustrated bone screws 30 are preferred for use with connectors 1 of the invention, particularly when the segment 7 is made from a material such as PEEK. Also, the bone screw 30, that includes upper and lower inserts as will be described in greater detail below, is preferred for attachment to the over-molded portion 5. A pair of bone screws 30, and/or other bone anchors and the connector 1 are then operably incorporated in the overall spinal implant system 32 for correcting degenerative conditions, deformities, injuries, or defects to the spinal column of a patient.

The bone screw assembly 30 includes a shank 34 that further includes a body 36 integral with an upwardly extending, substantially cylindrical upper end portion or capture structure 38; a receiver or head 40; a retaining and articulating structure 42; a first lower compression insert 44 and a second upper compression insert 46. The shank 34, the receiver 40, the retaining and articulating structure 42 and the first compression insert 44 are preferably assembled prior to implantation of the shank body 36 into a vertebra. FIGS. 2-8 further show a closure structure, generally 50 for capturing the longitudinal connector 1 within the receiver 40. Upon installation, which will be described in greater detail below, the closure structure 50 presses directly against the upper compression insert 46 in some applications, that in turn presses against the elastic portion 5 that in turn presses against the compression insert 44. As best shown in FIG. 7, the wrap-around inserts 44 and 46 limit pressure placed on any particular location of the surface 16 of the portion 5, providing uniform holding of the portion 5 without excessive compression and undue deformation thereof. As will be described in greater detail below, both the inserts 44 and 46 may include optional relieved areas to accommodate some deformation of the over-molded elastic portion 5. Furthermore, as will also be described in greater detail below, the closure structure 50 in some applications can also press directly against the lower insert 44 at surface 118, limiting the pressure interface between the inserts 46 and 44 and the elastic portion 5. The compression insert 44 in turn presses against the retaining and articulating structure 42 that is threadably mated or in other ways connected to the shank upper portion 38 to bias the retaining and articulating structure 42 into fixed frictional contact with the receiver 40, so as to substantially attach and orient the longitudinal connector 1 relative to the vertebra (not shown) and yet allow for relative movement of the outer portion 5 with respect to the inner core extension 8, providing relief (e.g., shock absorption) and protected movement with respect to torsion and distractive and compressive forces placed on connector 1 along the axis A. Again, the outer surface 16 of the portion 5 is also able to twist or turn with respect to the core 8, providing some relief for torsional stresses. However, the solid inner core 8 does not participate in or provide any means for torsional elasticity or axial compression and distraction along a length of the portion 5. The solid inner core does, however, provide support to the spine and resistance to shear.

The receiver 40, the shank 34, the retaining and articulating structure 42 and the compression inserts 44 and 46 cooperate in such a manner that the receiver 40 and the shank 34 can be secured at any of a plurality of angles, articulations or rotational alignments relative to one another and within a selected range of angles both from side to side and from front to rear, to enable flexible or articulated engagement of the receiver 40 with the shank 34 until both are locked or fixed relative to each other. Alternatively, it is foreseen that the connector 1 could involve the use of an upper compression insert in an open receiver that is integral or fixed in position with respect to a bone screw shank or bone hook, or that the receiver could have limited angular movement with respect to the shank, such as a hinged connection.

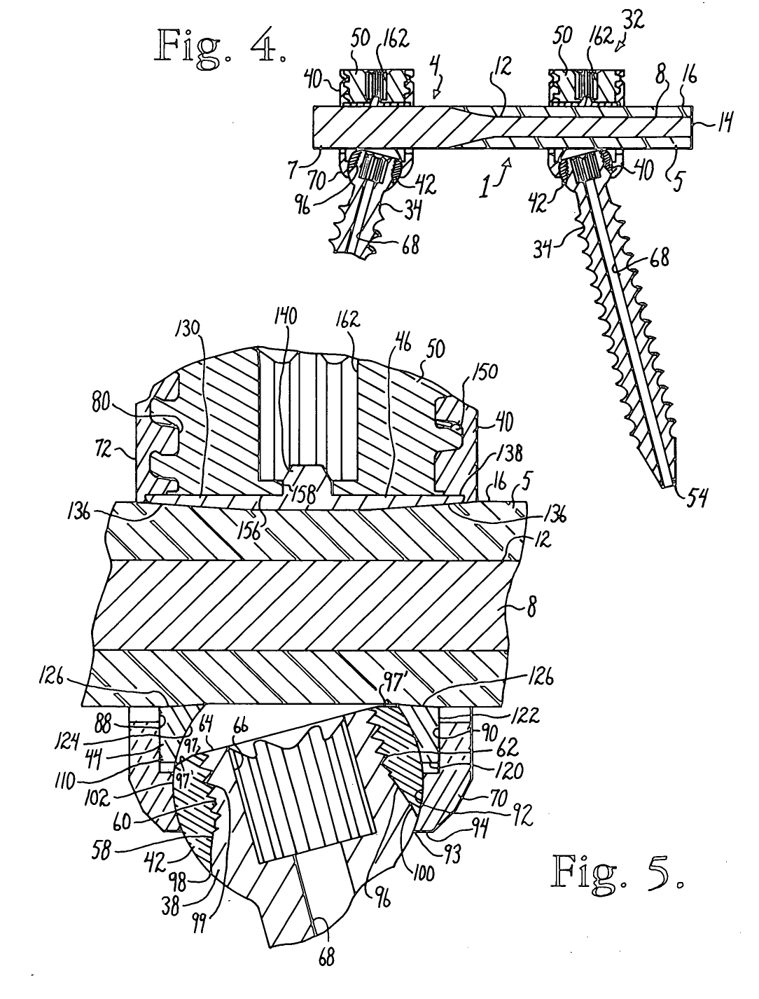

The shank 34 of the bone screw assembly 30, best illustrated in FIGS. 4-6, is elongate, with the shank body 36 having a helically wound, radially outwardly extending bone implantable thread axially extending from near a tip 54 of the body 36 to near a substantially cylindrical surface 56 located adjacent to the shank upper portion or capture structure 38. The shank upper portion 38 further includes a radially extending and slanted structure 58 providing a buttress stop feature for frictional engagement with and placement of the retaining and articulating structure 42. During use, the body 36 utilizing the outer thread thereon for gripping and advancement is implanted into the vertebra leading with the tip 54 and driven down into the vertebra with an installation or driving tool so as to be implanted in the vertebra to near the cylindrical surface 56.

To provide a biologically active interface with the bone, the shank body 36 may be coated, perforated, made porous or otherwise treated. The treatment may include, but is not limited to a plasma spray coating or other type of coating of a metal or, for example, a calcium phosphate; or a roughening, perforation or indentation in the surface of the body 36, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃(PO₄)₂, tetra-calcium phosphate (Ca₄P₂O₉), amorphous calcium phosphate and hydroxyapatite (Ca₁₀(PO₄)₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding. To improve the anchorage between the shank and vertebra, bone cement can be injected into the bone and the screw shank inserted thereafter. Such an approach can improve fixation in pathologic and weakened bone, such as that seen in patients with cancer and/or osteoporosis and osteopenia.

The sloped or slanted structure 58 extends radially outward and away from the cylindrical projection 56. Further extending axially from the sloped portion 58 is a cylindrical portion 60 having a helically wound guide and advancement structure, such as the illustrated V-thread 62 that provides a connective or capture apparatus disposed at a distance from the threaded shank body 36 and thus at a distance from the vertebra when the body 36 is implanted in the vertebra. Although a simple thread 62 is shown in the drawings, it is foreseen that other structures including other types of threads, such as buttress, square and reverse angle threads, and non threads, such as helically wound flanges with interlocking surfaces, may be alternatively used in place of the thread 62 in alternative embodiments of the present invention. An upper or top surface 64 is disposed substantially perpendicular to an axis of rotation B of the shank 34. An internal drive feature 66 is formed in the top surface 64. The driving feature or formation 66 is sized and shaped to cooperate with a driver for rotating and driving the shank body 36 into bone. Although a star-shaped feature is shown (such as that sold under the trademark TORX), it is foreseen that other driving features or apertures, such as hex, slotted, tri-wing, spanner, or the like may also be utilized according to the invention.

In the illustrated embodiment, the shank 34 is cannulated with a small central bore 68 extending an entire length of the shank along axis B. The bore 68 is coaxial with the threaded body 36, providing a passage through the shank interior for a length of wire or pin inserted into the vertebra prior to the insertion of the shank body 36, the wire or pin providing a guide for insertion of the shank body 36 into the vertebra.

Also with reference to FIGS. 4-6, the receiver 40 includes a base 70 integral with a pair of opposed upstanding arms 72 that extend from the base 70 to a top surface 74. The arms 72 form a U-shaped cradle and define a U-shaped channel 76 between the arms 72 and include an upper opening 77 and a lower seat 78 having substantially the same radius as the portions 5 and 6 of the longitudinal connector 1 for operably snugly receiving the connector 1.

Each of the arms 72 has an interior surface that defines an inner cylindrical profile and includes a partial helically wound guide and advancement structure 80. In the illustrated embodiment, the guide and advancement structure 80 is a partial helically wound flangeform configured to mate under rotation with a similar structure on the closure member 50, as described more fully below. However, it is foreseen that the guide and advancement structure 80 could alternatively be a buttress thread, a square thread, a reverse angle thread or other thread like or non-thread like helically wound advancement structures for operably guiding under rotation and advancing the closure 50 downward between the arms 72 and having such a nature as to resist splaying of the arms 72 when the closure 50 is advanced into the U-shaped channel 76.

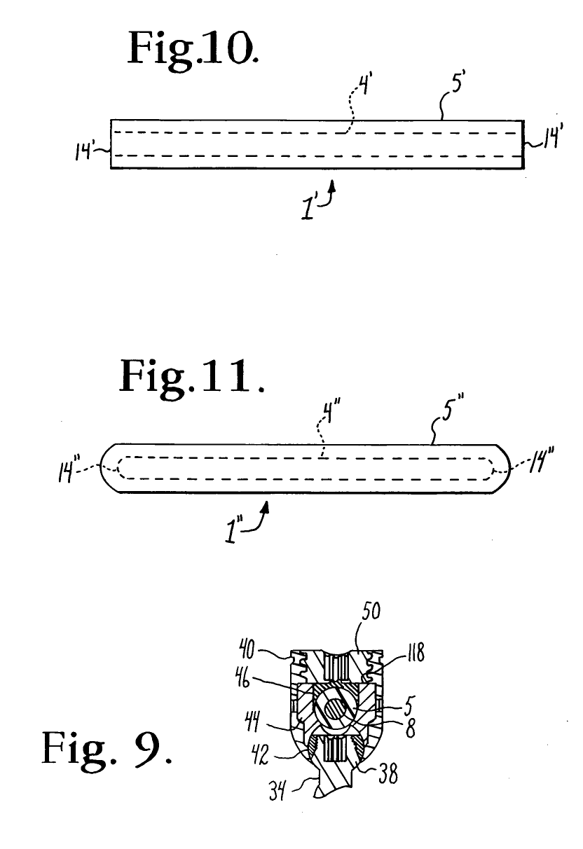

Each of the arms 72 includes a V-shaped or undercut tool engagement groove 82 formed on an outer surface thereof which may be used for holding the receiver 40 with a holding tool (not shown) having projections that are received within the grooves 82 during implantation of the shank body 36 into the vertebra. The grooves 82 may also cooperate with a holding tool during bone screw assembly and during subsequent installation of the connector 1 and closure 50. It is foreseen that tool receiving grooves or apertures may be configured in a variety of shapes and sizes and be disposed at other locations on the arms 72. Each of the arms 72 further include opposed apertures 84 that may be used for holding the receiver 40 and may also be used to access structure 85 for holding the lower insert 44 in alignment. With reference to FIGS. 8 and 9, the structure 85 may be in the form of material or a spring tab that is pushed or biased into a cooperating aperture 86 of the insert 44.

Communicating with the U-shaped channel 76 and located within the base 70 of the receiver 40 is a chamber or cavity 88 partially defined by an inner cylindrical surface 90 and a substantially spherical seating surface 92, the cavity 88 opening upwardly into the U-shaped channel 76. The base 70 further includes a restrictive neck 93 adjacent the seating surface 92. The neck 93 defines an opening or bore communicating with the cavity 88 and a lower exterior 94 of the base 70.

The neck 93 is sized and shaped to be smaller than a radial dimension of the retaining and articulating structure 42 so as to form a restriction at the location of the neck 93 relative to the retaining and articulating structure 42, to prevent the structure 42 from passing from the cavity 88 and out into the lower exterior 94 of the receiver 40 when the retaining and articulating structure 42 is seated on the seating surface 92. It is foreseen that the retaining and articulating structure could be compressible (such as where such structure has a missing section) and could be loaded through the neck 93 and then allowed to expand and fully seat in the spherical seating surface 92. Other bottom loading capture structures could be utilized.

With reference to FIGS. 4-6 and also to FIGS. 8 and 9, the retaining and articulating structure 42 has an operational central axis that is the same as the elongate axis B associated with the shank 34. The retaining and articulating structure 42 has a central bore 96 that passes entirely through the structure 42 from a substantially annular and planar top surface or upper rim 97 to a bottom surface or lower rim 98 thereof. The top surface 97 is adjacent to a ramped substantially planar surface portion 97′ disposed at an angle with respect to the top surface 97 and sized and shaped to clear and be spaced from the connector 1 at any angle of articulation of the bone screw shank 34 with respect to the receiver 40 as best shown in FIG. 5. Thus, the lower insert 44 is in direct engagement with the connector 1 and the retainer 42 does not touch the elastomeric portion 5 when the overall system 32 is fully assembled. An inner cylindrical surface defining a substantial portion of the retainer bore 96 has a helically wound guide and advancement structure thereon as shown by a v-shaped helical rib or thread 99 extending from adjacent the top surface 97 to a sloped surface 100 that is disposed between the thread 99 and the bottom surface 98. Although a simple helical rib 99 is shown in the drawings, it is foreseen that other helical structures including other types of threads, such as buttress and reverse angle threads, and non threads, such as helically wound flanges with interlocking surfaces, may be alternatively used in an alternative embodiment of the present invention. The inner thread 99 is configured to mate under rotation with the helical guide and advancement structure or thread 62 of the shank upper portion 38. The sloping surface 100 slidingly abuts against the slanted surface 58, providing frictional fixed contact between the retainer 42 and the shank upper portion 38 when the retainer 42 is tightened against the upper portion 38 upon mating rotation of the guide and advancement structures 62 and 99 until the top surface 97 is substantially planar to the top surface 64 of the shank upper end portion 38.

The illustrated retaining and articulating structure 42 has a radially outer partially spherically shaped surface 102 sized and shaped to mate with the spherically shaped seating surface 92 of the receiver and having a radius approximately equal to the radius associated with the surface 92. The retaining and articulating structure radius is larger than the radius of the neck 93 of the receiver 40. Although not required, it is foreseen that the outer partially spherically shaped surface 102 may be a high friction surface such as a knurled surface or the like. The illustrated retaining and articulating structure top surface 97 extends from the central bore 96 to the slanted surface 97′. The slanted surface 97′ is disposed between and adjacent to both the surface 97 and the outer curved surface 102. The illustrated top surface 97 is disposed perpendicular to an axis of rotation of the structure 42.

It is also foreseen that the retaining and articulating structure outer surface may be elliptical or ellipsoid in shape rather than spheroid in shape. Such an elliptical surface would be sized and shaped to contact and seat within a substantially spherical seating surface, such as the seating surface 92. The seating or bearing surface could also be tapered, conical, cylindrical or combinations of these surfaces. Such an ellipsoid structure may be attachable to the shank upper portion by threads, a pin, compression, or the like as previously described with respect to the substantially spherical retaining and articulating structure 42. Furthermore, it is foreseen that an ellipsoid retaining structure may be integral with the bone screw shank and may include threads that allow the ellipsoid to be threadably received into a base of a bone screw receiver. Again, it is foreseen that other types of retaining structure, articulating and not, could be used to keep the upper end of the shank contained within the receiver.

With reference to FIGS. 5-9, the lower compression insert 44 includes a lower body 110 of substantially circular cross-section integral with a pair of upstanding arms 112. The body 110 and arms 112 form a generally U-shaped, open, through-channel 114 having a partially U-shaped bottom seating surface 116 having a radius substantially conforming to an outer radius of the outer surfaces 16 and 17 of the respective connector portions 5 and 7 and thus configured to operably snugly engage such portions at the outer surfaces 16 and 17 thereof. The arms 112 disposed on either side of the channel 114 each include a top surface 118 that is substantially parallel to an annular bottom surface 120. The arms 112 are sized and shaped to cooperate and closely fit within the cylindrical surface 90 of the receiver 40 when in operation. The compression insert body 110 includes a substantially cylindrical outer surface 122 and an inner substantially spherical surface 124. The surface 124 opens into the U-shaped channel 114; thus, a central through-bore extends along a central axis of the compression structure 44 from the top surfaces 118 through the bottom surface 120. The surface 124 is sized and shaped to frictionally engage and mate with the outer spherical surface 102 of the retaining and articulating structure 42. The insert 44 is top loadable into the receiver 40 by aligning the insert 44 prior to loading with the arms 112 in the U-shaped channel 76. Then, once the arms 112 are disposed generally between the guide and advancement structure 80 and the receiver seating surface 92, the insert 44 is rotated about the axis B until the arms 112 are disposed directly beneath the guide and advancement structure 80. With particular reference to FIG. 5, the U-shaped seating surface 116 includes a pair of relieved surface areas 126. The surface areas 126 are disposed substantially centrally at a base of the seating surface 116 and slope outwardly and downwardly toward the cylindrical outer surface 122 and the bottom surface 120. The relieved areas 126 provide both pressure relief and accommodate viscoelastic movement of the over-molded portion 5, and, for example, the portion 7 when the portion 7 material is PEEK.

With reference to FIGS. 5-9, the upper or second compression insert 46 includes a substantially rectangular body 130 integral with a pair of downwardly extending legs 132 that form a generally U-shaped seating surface 134 having a radius substantially conforming to the outer radius of the surfaces 16 and 17 of the respective connector portions 5 and 7. The surface 134 is thus configured to operably snugly engage the portion 5 or portion 7 opposite the first or lower compression insert 44. As further illustrated in FIG. 7, outer substantially planar surfaces of the legs 132 of the insert 46 are received within the arms 112 of the insert 44 that define the through channel 114 providing substantial gripping by substantially wrapping around the connector portion 5 or 7 being received thereby. As will be discussed below, the relationship between the inserts 44 and 46 also desirably limits the pressure placed upon the elastic portion 5 while providing adequate gripping of the portion 5 over a substantial portion of the surface 16. With particular reference to FIG. 5, the U-shaped seating surface 134 also includes a pair of relieved surface areas 136. The surface areas 136 are substantially centrally located between the legs 132 and slope outwardly and upwardly toward an upper planar surface 138 of the insert body 130. In cooperation with the relieved areas 126, the relieved areas 136 provide both pressure relief and accommodate viscoelastic movement of the over-molded portion 5, and also, for example, the portion 7 when the portion 7 material is PEEK. The compression insert 46 further includes a pin 140 of substantially circular cross section disposed centrally on the top surface 138 and extending upwardly therefrom, being sized and shaped to fit within a central aperture of the closure 50 to be discussed more fully below.

With reference to FIGS. 3-9, the closure structure 50 can be any of a variety of different types of closure structures for use in conjunction with the present invention with suitable mating structure on the upstanding arms 72 of the receiver 40. The closure structure 50 is rotatable between the spaced arms 72, but could be a slide-in closure structure or a 90 degree twist-in structure or the like. The illustrated structure closure structure 50 is substantially cylindrical and includes an outer helically wound guide and advancement structure in the form of a flange form 150. The illustrated guide and advancement structure 150 operably joins with the guide and advancement structure 80 disposed on the interior of the arms 72. The guide and advancement structure 150 utilized in accordance with the present invention may take a variety of forms, including those described in Applicant's U.S. Pat. No. 6,726,689, which is incorporated herein by reference. It is also foreseen that according to the invention the guide and advancement structure 150 could alternatively be a buttress thread, a square thread, a reverse angle thread or other thread like or non-thread like helically wound advancement structure for operably guiding under rotation and advancing the closure structure 50 downward between the arms 72 and having such a nature as to resist splaying of the arms 72 when the closure structure 50 is advanced into the U-shaped channel 76.

The closure structure 50 includes a lower surface 156 having a central recess or bore 158 formed thereon. The bore 158 is substantially cylindrical having a central axis operationally coaxial with the receiver 40. The lower surface 156 is planar. The central recess 158 is sized and shaped to receive the pin 140 of the compression insert 46, with the lower surface 156 frictionally engaging the top planar surface 138 of the compression insert 46 when fully mated therewith, as illustrated in FIGS. 8 and 9. It is foreseen that the closure structure 50 could be used without the insert 46 and could have different bottom surfaces.

The closure 50 has a top surface 160 with an internal drive in the form of an aperture 162, illustrated as a star-shaped internal drive, for example, sold under the trademark TORX. A driving tool (not shown) sized and shaped for engagement with the internal drive 162 is used for both rotatable engagement and, if needed, disengagement of the closure 50 from the arms 72. Although a star-shaped internal drive 162 is shown in the drawings, the tool engagement structure may take a variety of tool-engaging forms and may include but is not limited to a hex shape or more than one aperture of various shapes. As best shown in FIG. 5, in the illustrated embodiment, the bore 158 communicates with the cavity formed by the internal drive 162, with the pin 140 being held in position by the annular surface defining the bore 158 and protruding into the cavity formed by the internal drive feature 162. It is also foreseen that the closure structure 50 may alternatively include a break-off head designed to allow such a head to break from a base of the closure at a preselected torque, for example, 70 to 140 inch pounds. Such a closure structure would also include a base having an internal drive to be used for closure removal.

Prior to the polyaxial bone screw assembly 30 being implanted in a vertebra (not shown), the retaining and articulating structure 42 is typically first inserted or top-loaded, into the receiver U-shaped channel 76, and then into the cavity 88 to dispose the structure 42 adjacent the inner seating surface 92 of the receiver 40. The shank upper portion 38 is preloaded, inserted or bottom-loaded into the receiver 40 at the bore defined by the neck 93. The retaining and articulating structure 42, now disposed in the receiver 40 is coaxially aligned with the shank upper portion 38 so that the helical v-shaped thread 80 rotatingly mates with the thread 99 of the retaining and articulating structure 42. The shank 34 and/or the retaining and articulating structure 42 are rotated to fully mate the structures 80 and 99, fixing the shank upper portion 38 to the retaining and articulating structure 42. At this time the shank 34 is in slidable and rotatable engagement with respect to the receiver 40, while the retaining and articulating structure 42 and the neck 93 of the receiver 40 cooperate to maintain the shank body 36 in rotational and pivotal relation with the receiver 40. The shank body 36 can be rotated through a substantial angular rotation relative to the receiver 40, both from side to side and from front to rear so as to substantially provide a universal or ball joint wherein the angle of rotation is only restricted by engagement of the shank body 36 with the neck 93 of the receiver 40.

In the embodiment shown, the compression insert 44 is then loaded into the receiver 40 with the arms 112 aligned with the receiver 40 U-shaped channel 76. The compression insert 44 is initially top or down-loaded into the receiver 40 until the arms 112 are disposed generally below the guide and advancement structure 80. The insert 44 is then rotated about the axis B until the arms 112 are disposed directly beneath the guide and advancement structure located on each arm 72, with the surface 124 being in sliding contact with the surface 102 of the retaining and articulating structure 42. At this time, tabs or other structure 85 of the receiver 40 may be biased or pressed against the insert 44 at the aperture 86 to retain the insert 44 in a desired position for receiving the connector 1, prohibiting further rotation of the insert 44 with respect to the axis B. To ready the assembly 30 for implantation into bone, the shank 34, the receiver 40 and the compression insert 44 central axes are all aligned along the axis B, providing access to the drive formation 66 on the shank upper portion 38 through the central bore formed in the compression insert 44.

The assembly 30 is then typically screwed into a vertebra by rotation of the shank 34 using a driving tool (not shown) with a driving formation that operably drives and rotates the shank 34 by engagement thereof with the shank at the driving formation 66.

At least two and up to a plurality of bone screw assemblies 30 are implanted into vertebrae for use with the longitudinal connecting member assembly 1. Each vertebra may be pre-drilled to minimize stressing the bone. Furthermore, when a cannulated bone screw shank is utilized, each vertebra will have a guide wire or pin (not shown) inserted therein that is shaped for the bone screw cannula 68 of the bone screw shank and provides a guide for the placement and angle of the shank 34 with respect to the vertebra. A further tap hole may be made and the shank body 36 is then driven into the vertebra by rotation of the driving tool (not shown). It is foreseen that the screws and the longitudinal connecting member can be inserted in a percutaneous or minimally invasive surgical manner.

With particular reference to FIGS. 1-4, the longitudinal connector 1 is formed in a factory setting with the segment 4 being held in a jig or other holding mechanism and an elastomeric polymer is molded about the inner core extension 8. The polymer flows about and, in some cases, firmly adheres to the extension 8, occurring for example, by chemical bonding or with the aid of an adhesive. The resulting molded portion 5 surrounds all or a substantial portion of the surface of the extension 8.

The connector 1 is eventually positioned in an open or percutaneous manner within the U-shaped channels 76 of two or more bone screw assemblies 30. The connector 1 can be straight, pre-bent or curvilinear and in some embodiments it can be cut to length. The second or upper compression insert 46 is then placed in each assembly 30 with the U-shaped seating surface 134 facing the portion 5 or the portion 7. The closure structure 50 is then inserted into and advanced between the arms 72. In some cases, the closure 50 and the insert 46 can be pre-assembled first. As the closure structure 50 is rotated between the arms 72, the central recess or aperture 158 receives the pin 140 of the compression insert 46, centering the insert 46 with respect to the receiver 40 and the connector 1. Continued rotation of the closure structure 50 results in engagement between the surfaces 16 and 17 of the respective portions 5 and 7 with both the seating surfaces 116 and 134, uniformly pressing the compression inserts 44 and 46 against the surface 16 or the surface 17. The seating surfaces of the inserts 44 and 46 both grip and cradle the over molded portion 5 from crushing against the inner core extension 8. Thus, although torquing of the closure structure 50 against the compression insert 46 clamps the portion 5 with enough force to keep the connector 1 in a fixed position in the receiver 40, the upper and lower compression inserts 44 and 46 do not compress the elastomeric portion 5 onto the core 8 with such force as to fix the surface 16 with respect to the core 8. Therefore, when a bone screw 30 is attached to the portion 5 and another bone screw 30 is attached to the portion 7, the cradle-like gripping of the inserts 44 and 46 allow the portion 5 to move with respect to the core 8 sufficient to provide elastic compression and distraction between the pair of bone screws 30. Also, as the closure 50 is driven into the receiver 40, the closure bottom surface 156 engages the lower insert 44 top surface 118 further limiting crushing of the elastomeric portion 5 against the core extension 8. With particular reference to FIG. 9, the engagement of the insert 44 with the closure 50 provides locking of the polyaxial mechanism independent of the pressure placed upon the elastic portion 5, fixing the retainer 42 firmly against the receiver inner surface 92. For example, about 40 to about 70 inch pounds of pressure are required for fixing the connector 1 in place without crushing the over-molded portion 5 against the core 8. However, about 80 to about 120 inch pounds pressure may be required for fixing the bone screw shank 34 with respect to the receiver 40. The cooperation between the compression inserts 44 and 46, cradling of the connector 1 there-between at the elastic-covered portion 5 as well as the cooperation between the closure 50 and the lower insert 44, allow for a total torquing of 80 to 120 inch pounds, with only 40 to 70 inch pounds of that force being placed on the portion 5. With reference to FIG. 8, the closure 50 presses the insert 46 against the hard rod outer surface 17 of the elongate portion 7 that in turn presses against the lower pressure insert 44, fixing the retainer 42 firmly against the receiver inner surface 92. As can be seen in FIG. 8, the closure 50 may be spaced from the surfaces 118 of the insert 44 when the portion 7 and the bone screw shank 34 are both locked into place. The connector 1 is substantially dynamically loaded and oriented relative to the cooperating vertebra, providing relief (e.g., shock absorption) and protected movement with respect to flexion, extension, distraction, compressive, torsion and shear forces placed on the connector 1 and the connected bone screws 30.

If removal of the connector 1 from any of the assemblies 30 is necessary, or if it is desired to release the connector 1 at a particular location, disassembly is accomplished by using the driving tool (not shown) with a star-shaped driving formation on the closure structure 50 internal drive 162 to rotate and remove the closure structure 50 from the receiver 40. Disassembly of the assembly 32 is accomplished in reverse order to the procedure described previously herein for assembly.

Eventually, if the spine requires more rigid support, the connecting member assembly 1 according to the invention may be removed and replaced with another longitudinal connecting member, such as a solid rod, having the same diameter as the end portions 5 and 7, utilizing the same bone screws 30 and the same or similar closure structures 50. Alternatively, if less support is eventually required, a less rigid, more flexible assembly, for example, a connector 1 made from a more flexible material or with a core 8 of smaller diameter may replace the connector 1, also utilizing the same bone screws 30.

With reference to FIG. 10 the reference numeral 1′ generally designates an alternative embodiment of a dynamic stabilization longitudinal connector according to the present invention. The connector 1′ includes an elongate inner core 4′ of substantially uniform cross-section and an elastic over-molded portion 5′. The elongate segment 4′ extends along an entire central longitudinal axis of the connector 1′ between a pair of opposed end surfaces 14′. In the illustrated embodiment, the connector 1′ is cylindrical and of uniform circular cross-section along an entire length thereof. The over-molded portion 5′ is molded and adhered onto the core 4′ along an entire length thereof and terminates at the core ends 14′. It is foreseen that according to other embodiments of the invention, the core 4′ and/or the over-molded portion 5′ may be of other cross-sectional geometries, including but not limited to oval, square, rectangular and other polygonal shapes. Mixtures of cross-section may also be utilized. The core 4′ and the portion 5′ are made from the materials previously described herein with respect to the respective segment 4 and portion 5 of the connector 1. Two or more bone screws 30 having upper and lower inserts 44 and 46, respectively are preferably utilized to firmly grip the connector 1′ at the portion 5′ without overly compressing or deforming the portion 5′ against the core 4′.

With reference to FIG. 11 the reference numeral 1″ generally designates another alternative embodiment of a dynamic stabilization longitudinal connector according to the present invention. The connector 1″ includes an elongate inner core 4″ of substantially uniform cross-section and an elastic over-molded portion 5″. The elongate segment 4″ extends along an entire central longitudinal axis of the connector 1″ between a pair of opposed end surfaces 14″. In the illustrated embodiment, the connector 1″ is cylindrical and of uniform circular cross-section along an entire length thereof. The over-molded portion 5″ is molded about the core 4″ along an entire length thereof and also completely over and about the end surfaces 14″. As the core 4″ is completely surrounded by the molded portion 5″, the portion 5″ does not need to otherwise be adhered to the core 4″ and, therefore, can slide against it. It is foreseen that according to other embodiments of the invention, the core 4″ and/or the over-molded portion 5″ may be of other cross-sectional geometries, including but not limited to oval, square, rectangular and other polygonal shapes. Mixtures of cross-section may also be utilized. The core 4″ and the portion 5″ are made from the materials previously described herein with respect to the respective segment 4 and portion 5 of the connector 1. Two or more bone screws 30 having upper and lower inserts 44 and 46, respectively are preferably utilized to firmly grip the connector 1″ at the portion 5″ without overly compressing or deforming the portion 5″ against the core 4″. It is foreseen that portion 5″ could be tubular and closed at one end. The tube could be slid onto the core 4″ and then sealed at the open end by a heating process or other sealing process.

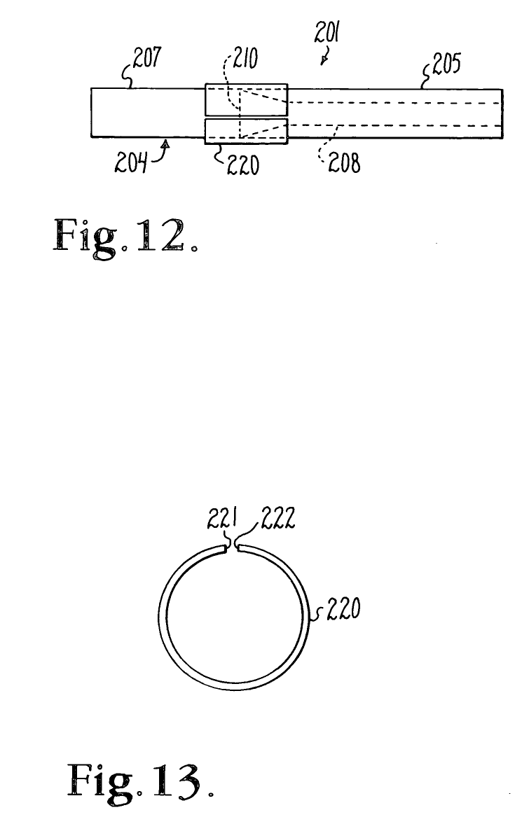

With reference to FIGS. 12 and 13, the reference numeral 201 generally designates another embodiment of a dynamic stabilization longitudinal connector according to the present invention. The connector 201 includes a substantially inelastic elongate segment, generally 204 and an elastic covering, illustrated by an over-mold 205 covering a portion of the segment 204. The illustrated elongate segment 204 further includes a first elongate portion 207 and an integral inner core extension 208 shown in phantom in FIG. 12. The segment 204 is identical or substantially similar in form and function to the segment 4 previously described herein with respect to the connector 1. The over-mold 205 is substantially similar to the over-mold 5 previously described herein with respect to the connector 1 with the exception that the over-mold 205 is fixed to the segment 204 at or about an edge 210 thereof by an open clamp 220. At the location 210, the cylindrical portion 207 tapers down to the core extension 208 of a smaller diameter than the portion 207 with the over-molded portion 205 being molded to the extension 208 starting at the location 210. The clamp 220 surrounds portions of both the over-mold 205 and the portion 207, pressing the over-mold 205 into fixed frictional engagement with the tapered surface of the portion 208. As illustrated in FIGS. 12 and 13, the clamp 220 is substantially cylindrical in shape, having opposed end surfaces 221 and 222 that provide an opening for slightly spreading the clamp 220 and placing the clamp about the connector 201 over the location 210. The clamp 220 may be made of a resilient material, such as a metal or metal alloy that snaps into place and presses against the over-mold 205 once the clamp is in position. Alternatively or additionally, a crimping tool (not shown) may be used to press the clamp 220 against the over-mold 205 and fix the over-mold against the elongate segment 204 near the location 210. It is foreseen that the clamp profile can be flush or tangent with the outer surface of portion 207 and portion 205, and that outer portion 205 can, again, be slid onto core member 208.

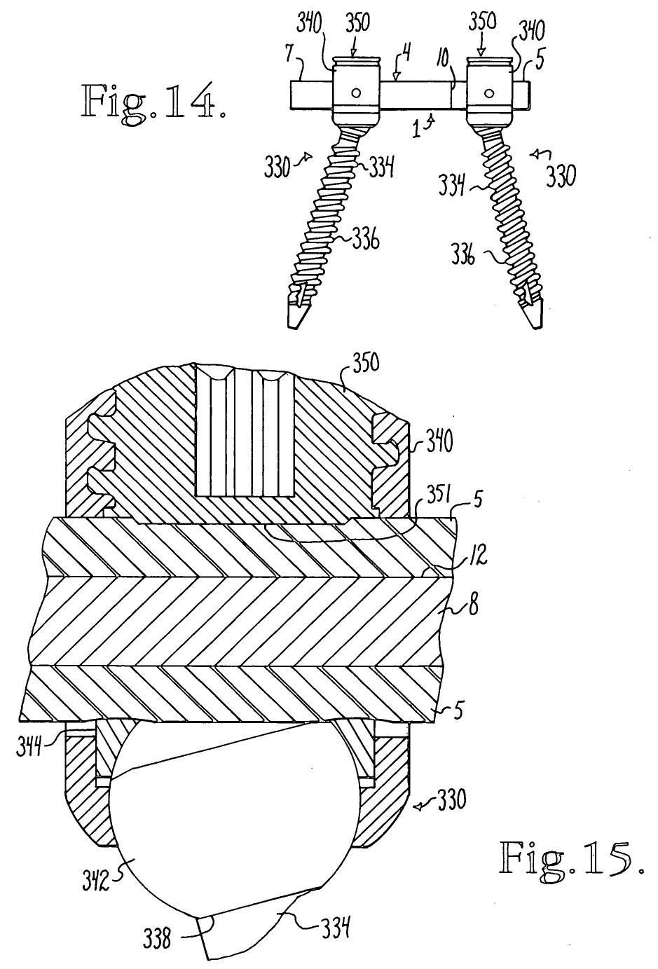

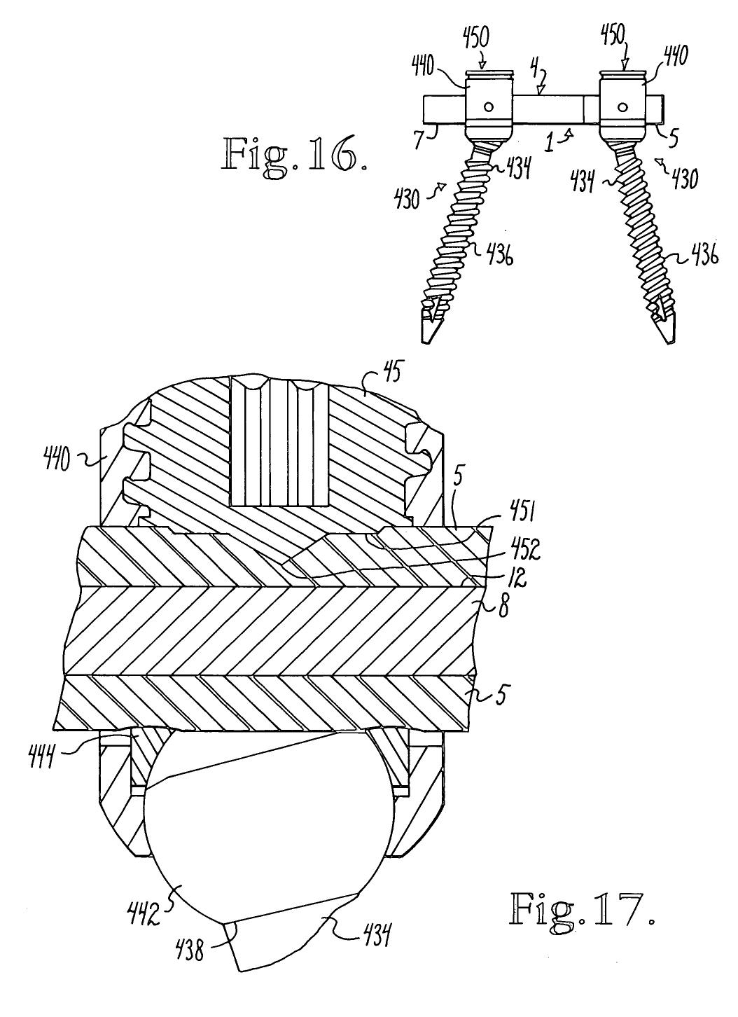

With reference to FIGS. 14 and 15, the connector 1 is shown with an alternative bone anchor assembly, generally 330 having a shank 334 with a shank body 336 and an upper end portion 338, a receiver 340, a retaining and articulating structure 342 and a lower pressure insert 344, the same or substantially similar to the respective bone screw 30 having the respective shank 34, shank body 36, upper end portion 38, receiver 40, retaining and articulating structure 42 and lower pressure insert 44 previously described herein. Unlike the bone screw 30, the bone screw 330 does not include the upper pressure insert 46. Rather, the assembly 330 includes a closure structure 350 that has a bottom surface 351 that directly engages the over-molded or slide-on portion 5, as illustrated in FIG. 15, or may directly engage the portion 7 (not shown). The illustrated surface 351 is substantially planar and projects from the base of the closure structure and into the over-molded or slide-on portion 5. In other embodiments, the surface 351 may be curved, include a roughening surface treatment, or may include one or more points or projections (see, e.g., FIGS. 16 and 17) to frictionally engage or partially pierce into the portion 5, but spaced from the core 8 such that the closure structure 350 does not fix against or in any way be in fixed spacial relation with the core 8. The closure structure 350 is otherwise substantially similar to the closure structure 50 previously described herein, having helical structure for rotatably mating with the receiver 340. Similar to the closure structure 50, the closure structure 350 engages the lower pressure insert 344 to lock the polyaxial bone screw mechanism. Specifically, as the closure structure 350 is rotated between the arms of the receiver 340 the surface 351 engages and presses against the portion 5 and deforms the polymer until the closure 350 engages the lower pressure insert 344. As shown in FIG. 15, the insert 344 also presses into and slightly deforms a portion of the polymer of the portion 5. Torquing of the closure structure 350 against the connector 1 clamps the portion 5 with enough force to keep the connector 1 captured within the receiver 340, but the closure 350 and the lower compression insert 344 do not compress the elastomeric portion 5 onto the core 8 with such force as to completely fix an entire radially directed thickness of the portion 5 with respect to the core 8. Therefore, when a bone screw 330 is attached to the portion 5 and another bone screw 30 or 330 is attached to the portion 7, the gripping of the insert 344 and the closure surface 351 allow the portion 5 to move with respect to the core 8 sufficient to provide elastic compression and distraction between the bone screw 330 and another bone screws 30 or 330. As the closure 350 is driven into the receiver 340, the closure bottom surface 151 engages the lower insert 344 top surface 118 further limiting crushing of the elastomeric portion 5 against the core extension 8, similar to what is shown in FIG. 9.

With reference to FIGS. 16 and 17, the connector 1 is shown with another alternative bone anchor assembly, generally 430 having a shank 434 with a shank body 436 and an upper end portion 438, a receiver 440, a retaining and articulating structure 442 and a lower pressure insert 444, the same or substantially similar to the respective bone screw 330 having the respective shank 334, shank body 336, upper end portion 338, receiver 340, retaining and articulating structure 342 and lower pressure insert 344 previously described herein with respect to FIGS. 14 and 15. The assembly 430 includes a closure structure 450 that is substantially similar to the closure structure 350 with the exception that a lower engagement surface 451 further includes a pointed projection 452 that is shown penetrating the polymer of the elastic portion 5. The projection 452 does not extend to the core 8, allowing for some of the polymer of the portion 5 to move with respect to the core 8.

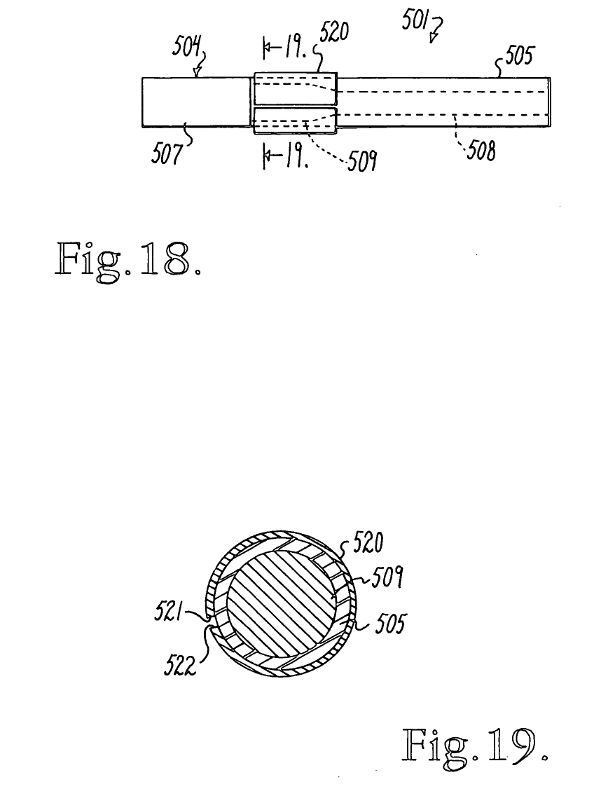

With reference to FIGS. 18 and 19, the reference numeral 501 generally designates another embodiment of a dynamic stabilization longitudinal connector according to the present invention. The connector 501 includes a substantially inelastic elongate segment, generally 504 and an elastic covering, illustrated by a slidable polymer sleeve 505 shown covering a portion of the segment 504. The illustrated elongate segment 504 further includes a first elongate portion 507 and an integral inner core extension 508 shown in phantom in FIG. 18. The core extension 508 further includes a mid-portion 509 of slightly smaller outer diameter than the portion 507. The portion 509 then tapers into the elongate extension 508 of even smaller diameter. The elastic sleeve 505 is formed and then slid onto the elongate segment 504, followed by clamping the sleeve 505 to the segment 504. The sleeve 505 is sized and shaped to include an inner surface closely slidingly cooperating with the portion 508. The illustrated sleeve 505 has an outer cylindrical surface with a diameter that is substantially the same as an outer diameter of the portion 507. As illustrated in FIGS. 18 and 19, the clamp 520 is also substantially cylindrical in shape, having opposed end surfaces 521 and 522 that provide an opening for slightly spreading the clamp 520 and placing the clamp about the connector 501 over the portion 509. The clamp 520 may be made of resilient material that presses against the elastomeric sleeve 505 once the clamp is in position. Alternatively or additionally, a crimping tool (not shown) may be used to press the clamp 520 against the elastomeric sleeve 505 and fix the sleeve 505 against the elongate segment 504. The clamp 520 can also be compressed around portion 509 and spot welded.

It is to be understood that while certain forms of the present invention have been illustrated and described herein, it is not to be limited to the specific forms or arrangement of parts described and shown.

What is claimed and desired to be secured by Letters Patent is as follows:

1. A longitudinal connecting member assembly having at least first and second bone anchors, the assembly comprising:
a) an inelastic elongate core having a first segment and a second segment in fixed relation to the first segment, the first bone anchor being attached to the assembly at the first segment; and
b) an elastomeric covering fixed to at least a portion of the second segment, the second bone anchor directly gripping the covering and spaced from the second segment, compression and distraction of the covering allowing movement of the second bone anchor with respect to the first bone anchor and with respect to the core.

2. The assembly of claim 1 wherein the second bone anchor includes first and second compression members, each compression member having an inner surface sized and shaped for direct engagement with the elastomeric covering, the first and second compression members cooperating to clamp only the elastomeric covering to the second bone anchor with the core being movable with respect to the first and second compression members.

3. The assembly of claim 2 wherein the first compression member is a lower compression member located adjacent a receiving surface of the bone anchor and the second compression member is an upper compression member located adjacent a closure top of the bone anchor.

4. The assembly of claim 3 wherein the upper compression member is at least partially received within the lower compression member.

5. The assembly of claim 2 wherein at least one of the first and second compression members has at least one relieved surface located adjacent the elastomeric covering.

6. The assembly of claim 1 wherein the first and second segments are integral.

7. The assembly of claim 1 wherein the first and second segments are both cylindrical, the first segment having an outer first diameter larger than an outer second diameter of the second segment, the elastomeric covering having a third outer diameter substantially equal to the first diameter of the first segment.

8. The assembly of claim 1 wherein the elastomeric covering is adhered to at least a portion of the second segment.

9. The assembly of claim 1 wherein the elastomeric covering is clamped to the second segment at a location between the first and second bone anchors.

10. The assembly of claim 1 wherein the second bone anchor includes a lower compression member having an inner surface sized and shaped for direct engagement with the elastomeric covering and a closure top, the lower compression member and the closure top cooperating to clamp only the elastomeric covering to the second bone anchor with the core being movable with respect to the lower compression member and the closure top.

11. The assembly of claim 10 wherein the closure top includes a projected surface engaging the elastomeric covering.

12. The assembly of claim 11 wherein the projected surface includes at least one point piercing into the elastomeric covering and spaced from the core.

13. In a medical implant assembly having at least two bone attachment structures cooperating with a longitudinal connecting member, the improvement wherein the longitudinal connecting member comprises:
a) first and second substantially inelastic segments, the second segment having an outer surface; and
b) an elastomer molded over the outer surface and firmly adhered to the outer surface.

14. The improvement of claim 13 wherein the first and second segments are integral.

15. The assembly of claim 13 wherein at least one of the bone attachment structures includes first and second compression members, each compression member having an inner surface sized and shaped for direct engagement with the elastomer, the first and second compression members cooperating to clamp only the elastomer to the at least one bone attachment structure, with the first and second segments being movable with respect to the first and second compression members.

16. The assembly of claim 15 wherein at least one of the first and second compression members has at least one relieved surface located adjacent the elastomer.

17. The assembly of claim 15 wherein the first and second segments are both cylindrical and coaxial, the first segment having an outer first diameter larger than an outer second diameter of the second segment, the elastomeric covering having a third outer diameter substantially equal to the first diameter of the first segment.

18. In a medical implant assembly including at least two bone attachment structures, the improvement comprising:
a) a longitudinal connecting member having
i) an inelastic core; and
ii) an outer polymeric covering fixed to a portion of the solid core; and
b) at least one of the bone attachment structures having first and second compression members, each compression member having an inner surface sized and shaped for frictional engagement with the polymeric covering, the first and second compression members cooperating to clamp only the polymeric covering to the bone attachment structure with the core remaining movable with respect to the first and second compression members.

19. A longitudinal connecting member assembly having at least first and second bone anchors, the assembly comprising:
a) a hard inelastic elongate core; and
b) an elastomeric covering completely surrounding the core at every surface thereof, the first and second bone anchors each attached to the covering, the covering being movable with respect to the core at the first and second bone anchors.

20. The assembly of claim 19 wherein the covering is adhered to the core.

21. The assembly of claim 19 wherein the first and second bone anchors each have opposed upper and lower compression inserts, each insert having an inner surface sized and shaped for direct engagement with the elastomeric covering along a surface thereof, the inserts cooperating to frictionally hold the elastomeric covering without fixing the covering against the core.

22. The assembly of claim 21 wherein the upper compression insert is at least partially received within the lower compression insert.

23. The assembly of claim 21 wherein at least one of the inserts has at least one relieved surface located adjacent the elastomeric covering.

24. In a medical implant assembly including at least two bone attachment structures, the improvement comprising:
a) a longitudinal connecting member having
i) a solid core; and
ii) an outer polymeric covering completely enveloping the solid core; and
b) each of the bone attachment structures having first and second compression members, each compression member having an inner surface sized and shaped for frictional engagement with the polymeric covering, the first and second compression members cooperating to clamp only the polymeric covering to the bone attachment structure with the solid core remaining movable with respect to the first and second compression members.
